(19) United States
(12) Patent Application Publication
(10) Pub. No.: US 2007/0134174 A1
(43) Pub. Date: Jun. 14, 2007

(54) Personal care composition

(76) Inventors: Christopher Irwin, Cincinnati, OH (US); Paul Robert Tanner, Lebanon, OH (US); Robert John Elsbrock, Cincinnati, OH (US); Richard John Szczepaniak, Liberty Twp, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/591,239

(22) Filed: Nov. 1, 2006

Related U.S. Application Data
(60) Provisional application No. 60/733,058, filed on Nov. 3, 2005.

(51) Int. Cl. A61K 8/81; A61K 8/73; A61K 8/64; A61K 8/29; A61K 8/49

(52) U.S. Cl. 424/59; 424/70.16; 424/70.14; 424/70.13

(57) ABSTRACT

Personal care composition comprising from about 5% to about 75% of an oil phase; an aqueous phase; from about 0.1% to about 2% of a polymeric thickener; and a skin care active; wherein said composition comprises at least two separate phases, and after agitation and dispensation from a suitable container containing a suitable propellant, forms stable foam.

### CROSS REFERENCE TO RELATED APPLICATION

This application claims the benefit of U.S. Provisional Application No. 60/733,058 filed Nov. 3, 2005.

### FIELD OF THE INVENTION

The present invention relates to a composition useful for regulating the condition of mammalian keratinous tissue, and methods of use thereof.

### BACKGROUND OF THE INVENTION

An ongoing need exists to provide personal care compositions which are both effective and have a pleasant appearance and feel. Foaming mousse compositions are desirable forms of personal care compositions, because they are easily applied and spread on the skin, and provide efficient distribution of active ingredients. Important aspects of foaming mousse compositions are that the dispensed foam is sufficiently thick and stable for a period of time sufficient to apply the composition. To this end, foaming mousse compositions typically contain emulsifiers and thickeners. However, emulsifiers and thickeners may contribute to an unpleasant feel on the skin, for example, an oily or a tacky feel. There exists a need, therefore, to provide mousse compositions which, when dispensed, provide stable foam, and when applied to the skin result in a pleasant feel.

### SUMMARY OF THE INVENTION

The present invention meets the aforementioned needs. Applicants have found that it is possible to make foaming mousse compositions which comprise lower levels of emulsifiers and thickeners, and thus have a more pleasant skin feel. In addition, the use of lower levels of emulsifiers and thickeners further provides a cost benefit. Prior to being dispensed, the compositions exhibit phase separation and thus, in this respect, are unstable. Typically, compositions that exhibit phase separation fail to produce stable foam upon dispensation. Applicants have unexpectedly found, however, that by combining appropriate amounts of select emulsifiers and thickeners in foaming mousse compositions, stable foam is dispensed in spite of the fact that the composition has separated phases prior to dispensation.

The following represent non-limiting embodiments of the present invention.

According to a first embodiment of the present invention, a personal care composition is provided comprising from about 5% to about 75% of an oil phase; an aqueous phase; from about 0.1% to about 2% of a polymeric thickener; and a skin care active; wherein said composition comprises at least two separate phases, and after agitation and dispensation from a suitable container containing a suitable propellant, forms stable foam. In an alternative embodiment, the composition is a sunscreen composition.

According to a second embodiment of the present invention, a method of regulating the condition of mammalian keratinous tissue, comprising the steps of providing a personal care composition according to the first embodiment, placing said composition in a suitable container with a suitable propellant; agitating said container sufficiently to produce stable foam upon dispensation from said container; dispensing a suitable amount of said composition; and topically applying said composition to mammalian keratinous tissue.

According to yet another embodiment of the present invention, a kit is provided, comprising a composition according to the first embodiment.

### DETAILED DESCRIPTION OF THE INVENTION

Whereas the specification concludes with claims that particularly point out and distinctly claim the present invention, it is believed that the invention will be better understood from the following details.

The present invention describes a personal care composition in the form of a foaming mousse, which exhibits phase separation prior to dispensation, and upon dispensation produces stable foam. The composition has an improved feel upon application to the skin relative to foaming mousse compositions which contain higher levels of emulsifiers and thickeners. The composition may be used in skin care, cosmetics, and hair care products, non-limiting uses of which include cleansers, moisturizers, conditioners, anti-aging compounds, exfoliants, peels, and combinations thereof. In one embodiment, the composition is applied to the face, neck and other exposed areas of the body. In one embodiment, the composition is a sunscreen composition.

In all embodiments of the present invention, all percentages are by weight of the total composition, unless specifically stated otherwise. All ratios are weight ratios, unless specifically stated otherwise. The number of significant digits conveys neither limitations on the indicated amounts nor on the accuracy of the measurements. All amounts indicating quantities, percentages, proportions, etc. are understood to be modified by the word “about” unless otherwise specifically indicated. All measurements are understood to be made at about 25° C. and at ambient conditions, where “ambient conditions” means conditions under about one atmosphere of pressure and at about 50% relative humidity.

Herein, “personal care composition” means compositions suitable for topical application on mammalian keratinous tissue. The personal care compositions described herein may contain one or more skin care actives. “Skin care actives,” or “actives,” as used herein, means compounds that aid in regulating the condition of skin and of other mammalian keratinous tissue, for example, by providing a benefit or improvement to the keratinous tissue.

Herein, “regulating the condition of keratinous tissue” means improving the condition of mammalian keratinous tissue and/or prophylactically regulating the condition of mammalian keratinous tissue, and includes, for example, protecting the tissue from ultraviolet radiation, and regulating the signs of skin aging. Herein, “improving the condition of mammalian keratinous tissue” means effecting a visually and/or tactilely perceptible positive change in the appearance and feel of the tissue. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

As used herein, “signs of skin aging,” include, but are not limited to, outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; flaking; dryness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Dermatologically-acceptable,” as used herein, means that the compositions or components thereof so described are suitable for use in contact with mammalian keratinous tissue without undue toxicity, incompatibility, instability, allergic response, and the like.

Herein, “unstable” or “instability” means that the unfoamed, or concentrated form of the composition, comprises at least two separate phases. “Separate,” as used herein, means visually and/or chemically distinguishable layers. Typically, the phases will be visually distinct without the aid of magnification, i.e. the composition will be visually perceived as non-homogenous. The phases may be aqueous and oil phases, and may be distinguishable on the basis of opacity, cloudiness, transparency, color or lack thereof, etc. When the composition is agitated (for example, vigorously shaken or otherwise mixed), the phases may temporarily cease to be separated. When the composition is allowed to remain substantially undisturbed at room temperature, the phases may once again become distinguishable. The phase separation may become noticeable after a few minutes or may take as long as several weeks, but typically will become noticeable after at least two days. Herein, “agitation” means a rapid, somewhat forceful back-and-forth motion, which results in mixing of the composition and in separated phases becoming substantially mixed. Agitation typically will be performed manually, but may be performed by mechanical means, by ultrasonic means, or by other equivalent means, and may be aided by the inclusion of solid objects that move freely in the container. When the composition of the present invention is sufficiently agitated prior to dispensation, the aerosol form of the composition is dispensed as stable foam. Herein, “dispensing” or “dispensation” means releasing a suitable amount of the aerosol composition from the container after agitation, onto one's hand, an implement, directly onto the keratinous tissue, or other desired location.

Herein, “suitable container” means a container suitable for containing the composition and a suitable propellant, alternatively under elevated pressure, for agitation, and for dispensing the composition in the form of a stable foam.

Herein, “suitable propellant” means a compound suitable for dispensing the composition as an aerosol, or stable foam. Examples of suitable propellants include, but are not limited to, hydrocarbons (for example, n-butane, isobutane, propane), chlorofluorocarbons, fluorocarbons (for example, 1,1,1,2,3,3,3-heptafluoropropane and 1,1,1,2-tetrafluoroethane), and mixtures thereof.

Herein, “stable foam” means that, after dispensation and left substantially undisturbed (e.g. prior to rubbing or exposing to other mechanical forces), the composition remains in the form of a foam for at least 10 seconds, alternatively 30 seconds, and alternatively 1 minute.

Herein, “delivery enhancement device” means any device that increases the amount of composition applied to and/or into the skin, more easily and/or efficiently delivers the composition, and/or increases the beneficial results derived from the composition, relative to that delivered without using the device.

Herein, “dietary supplement” means a dietary ingredient intended to supplement a regular diet, non-limiting examples of which include vitamins, minerals, herbs or other botanicals, amino acids, enzymes and metabolites. Herein, the dietary supplement is suitable for oral consumption and is administered orally. Examples of dietary supplements suitable for use in the present invention include, but are not limited to, vitamins and vitamin derivatives, peptides, essential fatty acids, and sugar amines. The form in which the dietary supplement is administered may vary widely, and includes, for example, tablets, capsules, gel tablets, and liquids. The dietary supplement further may be incorporated into a foodstuff or beverage.

Herein “kit” means a packaging unit comprising at least one composition described herein. The kit may comprise an outer packaging unit, which in turn may comprise one or more inner packaging units. The inner and outer packaging units may be of any type suitable for containing, presenting and/or reasonably protecting from damage the contents of the kit. The kit may comprise a plurality of components, such as a foaming mousse composition as described herein. The kit further may comprise one or more additional compositions, one or more orally ingestible dietary supplements, a delivery enhancement device, instructions for use of the device, instructions for complying with suitable application regimens, and combinations thereof.

A. Oil and Aqueous Phases

The composition of the present invention may comprise at least one oil phase. In one embodiment, the composition comprises from about 5% to about 75%, alternatively from about 8% to about 50%, and alternatively from about 10% to about 30% of the oil phase. Lipids and oils may be derived from animals, plants, or petroleum and may be natural or synthetic. The oil phase is understood to be immiscible in an aqueous phase, and may include natural and synthetic oils and other hydrophobic substances which exhibit limited solubility in an aqueous phase, including but not limited to oil-soluble ingredients, oil-soluble sunscreens and other oil-soluble skin care actives.

Suitable oil phase compounds include, but are not limited to, hydrocarbon oils and waxes, silicones, fatty alcohol and fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, salts, isomers and derivatives thereof, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, poly alpha olefins, hydrogenated polyisobutenes and combinations thereof.

Non-limiting examples of silicone oils suitable for use herein include dimethicone copolyol, silicone cross-polymers, dimethylpolysiloxane, diethylpolysiloxane, mixed C₁₋₃₀ alkyl polysiloxanes, phenyl dimethicone, dimethiconol, and combinations thereof. In one embodiment, the silicone oils are non-volatile silicone oils selected from the group consisting of dimethicone, dimethiconol, mixed C₁₋₃₀ alkyl polysiloxanes, silicone crosspolymers, and combinations thereof. These and other examples of silicone oils useful herein are described in U.S. Pat. No. 5,011,681, issued to Ciotti et al.

Non-limiting examples of silicone cross-polymers suitable for use herein include acrylate/bis-hydroxypropyl dimethicone crosspolymer, C₃₀₋₄₅ alkyl cetearyl dimethicone crosspolymer, acrylate/bis-hydroxypropyl dimethicone crosspolymer, C₃₀₋₄₅ alkyl cetearyl dimethicone crosspolymer, cetearyl dimethicone/vinyl dimethicone crosspolymer, dimethicone crosspolymer, dimethicone crosspolymer-3, dimethicone/phenyl vinyl dimethicone crosspolymer, dimethicone/vinyl dimethicone crosspolymer, diphenyl dimethicone crosspolymer, divinyldimethicone/dimethicone crosspolymer, trifluoropropyl dimethicone/trifluoropropyl divinyldimethicone crosspolymer, vinyl dimethicone/lauryl dimethicone crosspolymer, vinyldimethyl/trimethylsiloxysilicate stearyl dimethicone crosspolymer, polysilicone-11, and mixtures thereof.

The composition of the present invention further may comprise at least one aqueous phase. The aqueous phase may comprise water and/or other hydrophilic substances which exhibit limited solubility in an oil phase, including but not limited to water-soluble ingredients, water-soluble sunscreens and other water-soluble skin care actives.

B. Thickener

The composition of the present invention may comprise from about 0.1% to about 2%, and alternatively from about 0.5% to about 1.5%, of one or more thickening agents, including thickeners and gelling agents. Nonlimiting classes of thickening agents include crosslinked polyacrylate polymers and copolymers, hydrophobically-modified polyacrylate polymers and copolymers, polyacrylamide polymers and copolymers, polyacryloyldimethyl taurates, aminomethylpropanol (AMP)-based polymers and copolymers, polysaccharides and gums. In one embodiment, the composition of the present invention includes a thickening agent selected from hydrophobically-modified polyacrylate polymers and copolymers, polyacrylamide polymers and copolymers, aminomethylpropanol-based polymers and copolymers, and mixtures thereof. In one embodiment, the thickening agent is polyacrylamide polymers and copolymers. Non-limiting examples of suitable polymers include SEPIGEL® 305, SEPIPLUS® 400, SIMULGEL® NS and SIMULGEL® EG (all from Seppic, France), and PEMULEN® TR1 and TR2 (Noveon).

C. Skin Care Actives

The composition of the present invention may comprise at least one additional skin care active (“actives”), useful for regulating the condition of mammalian skin and other keratinous tissue. The actives may provide long-term, or chronic, benefits and/or more immediate benefits. Classes of suitable skin care actives include, but are not limited to vitamins, peptides and peptide derivatives, sugar amines, sunscreens, oil control agents, flavonoid compounds, antioxidants, preservatives, phytosterols, protease inhibitors, tyrosinase inhibitors, anti-inflammatory agents, and mixtures thereof. It should be noted, however, that many skin care actives may provide more than one benefit, or operate via more than one mode of action. Therefore, classifications herein are made for the sake of convenience and are not intended to limit the active to that particular application or applications listed.

1. Sunscreens

The composition of the present invention may comprise one or more sunscreen actives and/or ultraviolet (UV) light absorbers. Herein, “sunscreen” is understood to include both sunscreen actives and UV light absorbers. The sunscreen may be organic or inorganic, and may be water-soluble, oil-soluble, a particulate material which is insoluble in either an oil or an aqueous phase, and mixtures thereof. In one embodiment the composition of the present invention comprises a water-soluble and an oil-soluble sunscreen. In one embodiment, the composition may comprise from about 1% to about 30%, and alternatively from about 2% to about 20% by weight of the composition, of the sunscreen. Exact amounts will vary depending upon the chosen sunscreen and the desired Sun Protection Factor (SPF) and spectrum of protection (e.g. UV-A and/or UV-B), and are within the knowledge and judgment of one of skill in the art.

Examples of suitable sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxy-cinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy) propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimidazole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof.

2. Vitamins

The composition of the present invention may comprise one or more vitamins, for example, to provide antioxidant and/or other nutritional benefits to the skin. Herein, “vitamins” means vitamins, pro-vitamins, and their salts, isomers and derivatives. The vitamins may include water soluble vitamins, for example, vitamin B compounds (including B3 compounds such as niacinamide; nicotinic acid, C1-C18 nicotinic acid esters, and nicotinyl alcohol; B6 compounds, such as pyroxidine; and B5 compounds, such as panthenol, or “pro-B5”); and vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate; and mixtures thereof. The vitamins also may include those exhibiting limited solubility in water, such as vitamin A compounds, and all natural and/or synthetic analogs of Vitamin A, including retinoids, carotenoids, and other compounds which possess the biological activity of Vitamin A; vitamin D compounds; vitamin E compounds, or tocopherol, including tocopherol sorbate, tocopherol acetate, other esters of tocopherol; vitamin K compounds; and mixtures thereof. In one embodiment, the compositions of the instant invention may comprise from about 0.0001% to about 10%, alternatively from about 0.001% to about 8%, alternatively from about 0.01% to about 5%, and alternatively from about 0.1% to about 1%, of the vitamin.

3. Peptides and Peptide Derivatives

The composition of the present invention may comprise one or more peptides, for example, to aid in repair of skin, to aid in exfoliation, and to deliver other benefits to the skin. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®).

The composition may comprise from about 1×10⁻⁷% to about 20%, alternatively from about 1×10⁻⁶% to about 10%, and alternatively from about 1×10⁻⁵% to about 5% of the peptide.

4. Sugar Amines

The composition of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-D-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

5. Oil Control Agents

The composition of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.1% to about 2%, of an oil control agent.

6. Flavonoids

The composition of the present invention may comprise a flavonoid, for example, to provide anti-oxidation benefits. The flavonoid can be synthetic materials or obtained as extracts from natural sources, which also further may be derivatized. Examples of classes of suitable flavonoids are disclosed in U.S. Pat. No. 6,235,773, issued to Bissett, and include, but are not limited to, unsubstituted flavanone, methoxy flavanones, unsubstituted chalcone, 2′,4-dihydroxy chalcone, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin and glucosyl hesperidin, isoflavones such as soy isoflavones, including but not limited to genistein, daidzein, and equol, their glucosyl derivatives, and mixtures thereof.

The composition of the present invention may comprise from about 0.01% to about 20%, alternatively from about 0.1% to about 10%, and alternatively from about 0.1% to about 5% of flavonoids.

7. Other Skin Care Actives

The composition of the present invention may comprise non-vitamin antioxidants, preservatives, phytosterols and/or plant hormones, protease inhibitors, tyrosinase inhibitors, anti-inflammatory agents and N-acyl amino acid compounds.

Suitable non-vitamin antioxidants include, but are not limited to, BHT (butylated hydroxy toluene), L-ergothioneine (available as THIOTANE™); tetrahydrocurcumin, cetyl pyridinium chloride, camosine, diethylhexyl syrinylidene malonate (available as OXYNEX™), ubiquinone (co-enzyme Q10), and combinations thereof.

Suitable examples of plant sterols and/or plant hormones include, but are not limited to, sitosterol, stigmasterol, campesterol, brassicasterol, kinetin, zeatin, and mixtures thereof.

Suitable protease inhibitors include, but are not limited to, hexamidine, vanillin acetate, menthyl anthranilate, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France).

Other useful skin care actives include dehydroepiandrosterone (DHEA), its analogs and derivatives; alpha- and beta-hydroxyacids, including glycolic acid and octanoyl salicylate, arbutin, dimethyl aminoethanol (DMAE), kojic acid, dihydroxy acetone (DHA), soy proteins and peptides (for example, protease inhibitors such as soybean trypsin inhibitor, and Bowman-Birk inhibitor), arbutin, their isomers, salts, and derivatives, and mixtures thereof.

D. Surfactants

The composition of the present invention may comprise one or more surfactants. In one embodiment, the composition comprises from about 0.01% to about 1% of surfactant. Surfactants useful herein include those selected from the group consisting of anionic surfactants, amphoteric surfactants, zwitterionic surfactants, cationic surfactants, nonionic surfactants and mixtures thereof. In one embodiment, the surfactant is a nonionic surfactant. Non-limiting examples of suitable surfactants are disclosed in U.S. Pat. No. 5,624,666, issued to Coffindaffer et al.; in McCutcheon's, Detergents and Emulsifiers, North American edition (1986), published by Allured Publishing Corporation; and in McCutcheon's Functional Materials, North American Edition (1992).

E. Particulates

The composition of the present invention may comprise a particulate material. In one embodiment, the composition may comprise from about 0.5% to about 10% of a particulate material, and alternatively from about 1% to about 5% of a particulate material. Non-limiting examples of suitable particulate materials can be found in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2728. Other suitable particulate materials include, but are not limited to almond meal, aluminum oxide, apricot seed powder, bismuth oxychloride, boron nitride, cellulose and cellulose derivatives, clay, calcium oxide, inorganic salts, for example salts of carbonates and chlorides, iron oxide, jojoba seed powder, loofah, mica, peach pit powder, pecan shell powder, polyethylene, polybutylene, polyisobutylene, polymethylstyrene, polypropylene, polystyrene, polyurethane, nylon, polytetrafluoroethylene, polyhalogenated olefins, pumice, rice bran, sericite, silk, synthetic hectorite, titanium dioxide, tricalcium phosphate, and mixtures thereof. Also useful are particles made from mixed polymers (e.g., copolymers, terpolymers, etc.), among such are polyethylene/polypropylene copolymer, polyethylene/propylene/isobutylene copolymer, polyethylene/styrene copolymer, and mixtures thereof. Typically, the polymeric and mixed polymeric particles are treated via an oxidation process, for example to destroy impurities. The polymeric and mixed polymeric particles can also optionally be cross linked with a variety of common crosslinking agents, non-limiting examples including butadiene, divinyl benzene, methylenebisacrylamide, allyl ethers of sucrose, allyl ethers of pentaerythritol, and mixtures thereof. Other examples of useful particles include waxes and resins such as paraffins, carnuba wax, ozekerite wax, candellila wax, and urea-formaldehyde resins. When such waxes and resins are used herein it is important that these materials are solids at ambient and skin temperatures.

Other examples of particulate materials useful in the present invention include colored and uncolored pigments, interference pigments, inorganic powders and organic powders other than those described above, composite powders, optical brightener particles, and mixtures thereof. The average size of such particulates in general may be smaller than the aforementioned particulate materials, ranging for example from about 0.1 microns to about 100 microns. These particulates can, for example, be platelet shaped, spherical, elongated or needle-shaped, or irregularly shaped, surface coated or uncoated, porous or non-porous, charged or uncharged, and can be added to the current compositions as a powder or as a pre-dispersion. These particulate materials can be derived from natural and/or synthetic sources.

Suitable organic powders particulate materials include, but are not limited, to spherical polymeric particles chosen from the methylsilsesquioxane resin microspheres, for example, Tospearl™ 145A, (Toshiba Silicone); microspheres of polymethylmethacrylates, for example, Micropearl™ M 100 (Seppic); the spherical particles of crosslinked polydimethylsiloxanes, for example, Trefil™ E 506C or Trefil™ E 505C (Dow Corning Toray Silicone); sphericle particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminium starch octenylsuccinate, for example Dry FlO™ (National Starch); microspheres of polyethylene, for example Microthene™ FN510-00 (Equistar), silicone resin, polymethylsilsesquioxane silicone polymer, platelet shaped powder made from L-lauroyl lysine, and mixtures thereof.

Also useful herein are interference pigments. Herein, “interference pigments” means thin, platelike layered particles having two or more layers of controlled thickness. The layers have different refractive indices that yield a characteristic reflected color from the interference of typically two, but occasionally more, light reflections, from different layers of the platelike particle. The most common examples of interference pigments are micas layered with about 50-300 nm films of TiO₂, Fe₂O₃, silica, tin oxide, and/or Cr₂O₃. Such pigments often are pearlescent. Pearlescent pigments reflect, refract and transmit light because of the transparency of pigment particles and the large difference in the refractive index of mica platelets and, for example, the titanium dioxide coating. Interference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

Other pigments useful in the present invention can provide color primarily through selective absorption of specific wavelengths of visible light, and include inorganic pigments, organic pigments and combinations thereof. Examples of such useful inorganic pigments include iron oxides, ferric ammonium ferrocyanide, manganese violet, ultramarine blue, and chromium oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO₂, ZnO, or ZrO₂, are commercially available from a number of sources, for example, TRONOX TiO₂ series, SAT-T CR837, a rutile TiO2 (U.S. Cosmetics). Also suitable are charged dispersions of titanium dioxide, disclosed in U.S. Pat. No. 5,997,887, issued to Ha et al.

In one embodiment, the particulate material is selected from the group consisting of spherical polymeric particulates, TiO₂, interference pigments, and combinations thereof.

F. Methods of Use

The present invention provides for a method for regulating the condition of mammalian keratinous tissue, including but not limited to a method for improving the condition of mammalian keratinous tissue, prophylactically regulating the condition of mammalian keratinous tissue, and/or regulating the signs of skin aging. Any part of the external portion of the skin can be treated. In one embodiment, the composition is applied to the face and the neck.

Prior to dispensing from the container, the composition should be sufficiently mixed, for example, by vigorously agitating the container. The composition may be agitated for at least 3 seconds, alternatively for at least 10 seconds, and alternatively for at least 20 seconds. After agitation, a suitable amount of the composition may be dispensed. The composition may be applied directly to the keratinous tissue, or the composition may be applied to the palms of the hands, the fingers, or to an implement (e.g., a cotton ball, swab, pad, etc.). The composition further may be used in combination with a delivery enhancement device, non-limiting examples of which include an implement, such as a sponge or sponge-tipped applicator, a spray applicator, a brush, and combinations thereof. The composition typically is applied while still foamy, and may be rubbed into the skin to facilitate absorption.

The amount of the composition applied, the frequency of application and the period of use will vary widely depending upon the level of components of a given composition and the desired effect. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for one day.

G. Kit

The present invention further may comprise a kit, wherein the kit comprises a composition as described herein. The kit further may comprise one or more additional compositions, instructions for applying the composition(s), instructions for complying with a suitable application regimen, an implement, a substrate, a delivery enhancement device, a dietary supplement, and combinations thereof. The kit may comprise an outer packaging unit, which in turn may comprise one or more smaller, inner packaging units. The inner packaging units may comprise one or more of the individual components of the kit. The inner and outer packaging units may be of any type suitable for containing, presenting and/or reasonably protecting from damage the contents of the kit. The inner packaging units each may contain a quantity of a composition suitable for use in a single application regimen.

### EXAMPLES

### Examples 1-3

### Personal Care Compositions are Prepared from the Following Components

| | Example 1 | Example 2 | Example 3 |
|---|---|---|---|
| Water Phase: | | | |
| Water | qs | qs | qs |
| Glycerin | 7.0 | 5.0 | 5.0 |
| Disodium EDTA | 0.1 | 0.1 | 0.1 |
| Methylparaben | 0.2 | 0.2 | 0.2 |
| Niacinamide | 4.0 | 4.0 | 4.0 |
| D-panthenol | 0.5 | 0.5 | 0.5 |
| Phenylbenzimidazole Sulfonic | 1.0 | 1.0 | 1.0 |
| Acid | | | |
| Benzyl alcohol | 0.25 | 0.25 | 0.25 |
| Triethanolamine | 0.64 | 0.64 | 0.64 |
| Pentadecalactone | 0.05 | 0.05 | 0.05 |
| N-acetyl glucosamine | 2.0 | 2.0 | 2.0 |
| Oil Phase: | | | |
| Isopropyl Isostearate | 1.33 | 1.33 | 1.33 |
| Octisalate | 4.0 | 4.0 | 4.0 |
| Octocrylene | 1.0 | 1.0 | 1.0 |
| Avobenzone | 2.0 | 2.0 | 2.0 |
| Vitamin E Acetate | 0.1 | 0.1 | 0.1 |
| Cab-O-Sil HS5¹ | 0.25 | — | — |
| Ethylparaben | 0.2 | 0.2 | 0.2 |
| Propylparaben | 0.1 | 0.1 | 0.1 |
| Thickener: | | | |
| Sepigel 305² | 1.5 | 1.5 | 1.5 |
| Additional Ingredients: | | | |
| Microthene FN510³ | 1.0 | 1.0 | 1.0 |
| KTZ Interfine ™ Gold⁴ | 0.25 | 0.25 | — |
| KTZ Interfine ™ Red⁴ | 0.25 | 0.25 | — |
| KTZ Interfine ™ Green⁴ | 0.25 | 0.25 | — |
| KTZ Interfine ™ Blue⁴ | 0.25 | 0.25 | — |
| Polysorbate 20 | 0.5 | 0.5 | 0.5 |
| Dow Corning ™ 1503⁵ | 2.0 | 2.0 | 2.0 |
| Total: | 100% | 100% | 100% |

¹Fumed silica from Degussa ™
²Polyacrylamide, C13-14 isoparaffin, and laureth-7 from Seppic ™
³Polyethylene homopolymer spheres from Equistar ™.
⁴Titanium dioxide coated mica available from Kobo Products ™ Inc.
⁵Dimethicone and dimethiconol from Dow Corning ™

In a suitable vessel, the water phase ingredients are combined and heated to 75° C. In a separate suitable vessel, the oil phase ingredients are combined and heated to 75° C. Next the oil phase is added to the water phase and the resulting emulsion is milled (eg., with a Tekmar T-25). The thickener is then added to the emulsion and the emulsion is cooled to 45° C. while stirring. At 45° C., the interference pigments and remaining ingredients are added. The product is then cooled with stirring to 30° C., milled again, and then poured into suitable containers. The resulting formulations show visibly separate phases after at least two days at room temperature.

To make foaming mousses, 95% of each example formulation is combined with 5% A70 propellant (isobutane/propane blend) in a suitable aerosol container. After shaking for approximately 10 seconds, dispensing each aerosol formulation produced foam that was stable for at least 10 seconds.

### Examples 4-6

### Personal Care Compositions are Prepared by the Same Procedure as Examples 1-3, from the Following Components

| | Example 4 | Example 5 | Example 6 |
|---|---|---|---|
| Water Phase: | | | |
| Water | qs | qs | qs |
| Glycerin | 5.0 | 5.0 | 5.0 |
| Disodium EDTA | 0.1 | 0.1 | 0.1 |
| Methylparaben | 0.2 | 0.2 | 0.2 |
| Niacinamide | 4.0 | 4.0 | 4.0 |
| D-panthenol | 0.5 | 0.5 | 0.5 |
| Phenylbenzimidazole Sulfonic | 1.0 | 1.0 | 1.0 |
| Acid | | | |
| Benzyl alcohol | 0.25 | 0.25 | 0.25 |
| Triethanolamine | 0.64 | 0.64 | 0.64 |
| Pentadecalactone | 0.05 | 0.05 | 0.05 |
| N-acetyl glucosamine | 2.0 | 2.0 | 2.0 |
| Oil Phase: | | | |
| Isopropyl Isostearate | 1.33 | 1.33 | 1.33 |
| Octisalate | 4.0 | 4.0 | 4.0 |
| Octocrylene | 1.0 | 1.0 | 1.0 |
| Avobenzone | 2.0 | 2.0 | 2.0 |
| Vitamin E Acetate | 0.1 | 0.1 | 0.1 |
| Ethylparaben | 0.2 | 0.2 | 0.2 |
| Propylparaben | 0.1 | 0.1 | 0.1 |
| Cetearyl Glucoside/Cetearyl | 0.2 | 0.2 | — |
| Alcohol¹ | | | |
| PEG-100 stearate | 0.1 | 0.1 | — |
| Thickener: | | | |
| Sepigel ™ 305² | 3.5 | — | — |
| Additional Ingredients: | | | |
| Microthene FN510³ | 1.0 | 1.0 | 1.0 |
| Polysorbate 20 | 0.5 | 0.5 | 0.5 |
| Dow Corning ™ 1503⁴ | 2.0 | 2.0 | 2.0 |
| Total: | 100% | 100% | 100% |

¹Emulgade ™ PL68/50 from Cognis ™
²Polyacrylamide, C13-14 isoparaffin, and laureth-7 from Seppic ™
³Polyethylene homopolymer spheres from Equistar ™
⁴Dimethicone and dimethiconol from Dow Corning ™

Example 4 is similar to Example 3, except for the addition of an emulsifier system (cetearyl glucoside/cetearyl alcohol, and PEG-100 stearate) and additional polymeric thickener (Sepigel™ 305). In contrast to formulation example 3, which exhibits separate phases at room temperature, Example 4 is visibly stable (homogenous) at room temperature. Aerosol foaming mousses created with both Examples 3 and 4 produce foams that are stable for at least 10 seconds. However, Applicants believe that formulation Example 4 has a heavier and tackier skin feel than Example 3. Thus, by reducing the level of, or alternatively eliminating, stabilizing emulsifiers and stabilizing polymeric thickeners (comparing Example 4 and Example 3), stable foam is produced which has improved skin feel.

Examples 5 and 6 both show visibly separate phases after at least two days at room temperature. However, when combined with propellant (5% A70) in a suitable container, foam is produced which is unstable as defined herein (i.e. collapses less than about 10 seconds after dispensation).

The following table summarizes the stability, foaming behavior, and skin feel of Examples 1 through 6:

| | Examples 1-3 | Example 4 | Examples 5-6 |
|---|---|---|---|
| Composition Stability | Visibly | Stable (no | Visibly |
| | separates on | visible | separates on |
| | standing | separation) | standing |
| Mousse Foam Stability | Stable | Stable | Unstable |
| Composition Skin Feel | Improved | Worse | Improved |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) from about 5% to about 75% of an oil phase;
b) an aqueous phase;
c) from about 0.1% to about 2% of a polymeric thickener; and
d) a skin care active;
wherein said composition comprises at least two separate phases, and after agitation and dispensation from a suitable container containing a suitable propellant, forms stable foam.

2. The personal care composition of claim 1, further comprising a surfactant.

3. The personal care composition of claim 2, wherein said surfactant is nonionic.

4. The personal care composition of claim 1, further comprising from about 0.5% to about 10% of a particulate material selected from the group consisting of spherical polymeric particulates, TiO₂, interference pigments, and mixtures thereof.

5. The personal care composition of claim 1, wherein said polymeric thickener is selected from the group consisting of polyacrylamide polymers and copolymers, aminomethylpropanol-based polymers and copolymers, hydrophobically-modified polyacrylate polymers and copolymers, and mixtures thereof.

6. The personal care composition of claim 5, wherein said polymeric thickener is polyacrylamide polymers and copolymers.

7. The personal care composition of claim 1, wherein said skin care active is a sunscreen.

8. The personal care composition of claim 7, wherein said sunscreen is selected from the group consisting of oil-soluble sunscreens, water-soluble sunscreens, and mixtures thereof.

9. The personal care composition of claim 1 further comprising at least one additional skin care active.

10. The personal care composition of claim 9, wherein said skin care active is selected from the group consisting of vitamin B compounds, vitamin C compounds, vitamin E compounds, peptides, sugar amines, oil control agents, and combinations thereof.

11. The personal care composition of claim 10, wherein said skin care active is selected from the group consisting of niacinamide, palmitoyl-lysine-threonine, palmitoyl-lysine-threonine-threonine-lysine-serine, N-acetyl-D-glucosamine, salicylic acid, dehydroacetic acid, sodium dehydroacetate, and combinations thereof.

12. A personal care composition comprising:
a) from about 5% to about 75% of an oil phase;
b) an aqueous phase;
c) from about 0.5% to about 1.5% of polyacrylamide polymers and copolymers; and
d) a sunscreen;
wherein said composition comprises at least two separate phases, and after agitation and dispensation from a suitable container containing a suitable propellant, forms stable foam.

13. The personal care composition of claim 12, further comprising a surfactant.

14. The personal care composition of claim 13, wherein said surfactant is nonionic.

15. The personal care composition of claim 12, wherein said sunscreen is selected from the group consisting of oil-soluble sunscreens, water-soluble sunscreens, and mixtures thereof.

16. The personal care composition of claim 12 further comprising at least one additional skin care active.

17. The personal care composition of claim 16, wherein said additional skin care active is selected from the group consisting of vitamin B compounds, vitamin C compounds, vitamin E compounds, peptides, sugar amines, oil control agents, and combinations thereof.

18. The personal care composition of claim 17, wherein said skin care active is selected from the group consisting of niacinamide, palmitoyl-lysine-threonine, palmitoyl-lysine-threonine-threonine-lysine-serine, N-acetyl-D-glucosamine, salicylic acid, dehydroacetic acid, sodium dehydroacetate, and combinations thereof.

19. A method of regulating the condition of mammalian keratinous tissue, comprising the steps of:
a) providing a personal care composition comprising:
i. from about 5% to about 75% of an oil phase;
ii. an aqueous phase;
iii. from about 0.1% to about 2% of a polymeric thickener; and
iv. a skin care active;
wherein said composition comprises at least two separate phases;
b) placing said composition in a suitable container;
c) adding a suitable propellant to said composition;
d) agitating said container sufficiently to produce stable foam upon dispensation from said container;
e) dispensing a suitable amount of said composition;
f) topically applying said composition to mammalian keratinous tissue.

20. A kit comprising:
a) a personal care composition comprising:
i. from about 5% to about 75% of an oil phase;
ii. an aqueous phase;
iii. from about 0.1% to about 2% of a polymeric thickener; and
iv. a skin care active;
wherein said composition comprises at least two separate phases, and after agitation and dispensation from a suitable container containing a suitable propellant, forms stable foam;
b) at least one additional personal care composition.
